(19) United States
(12) Patent Application Publication
(10) Pub. No.: US 2011/0318818 A1
(43) Pub. Date: Dec. 29, 2011

(54) STRAW FOR THE CONSERVATION OF A PREDETERMINED DOSE OF A LIQUID BASE SUBSTANCE, IN PARTICULAR A BIOLOGICAL SUBSTANCE

(75) Inventors: Christian Beau, Voisins le Bretonneux (FR); Agnés Camus, Sainte Gauburge Sainte Colombe (FR); Anke Tusche, Rietschen (DE)

(73) Assignee: IMV Technologies, Saint Ouen Sur Iton (FR)

(21) Appl. No.: 13/141,025

(22) PCT Filed: Dec. 8, 2009
(86) PCT No.: PCT/IB09/55579

§ 371 Date: Sep. 12, 2011

(30) Foreign Application Priority Data
Dec. 18, 2008 (EP) 08291213.0

(51) Int. Cl. C12M 3/00

(52) U.S. Cl. 435/284.1; 435/307.1

(57) ABSTRACT

The straw comprises a tube (41) and a plug (42) consisting of a one-piece cylinder of microporous material, permeable to gases and liquid tight. The microporous material is a sintered self-sealing microporous material with at least a solid portion (42) having a diameter matching the inner diameter of said tube (41), said solid portion being in direct contact with said tube (41), said plug having in the tube a permeability to gases when it is dry such that if one end of the tube is placed in communication with a pressure source 150 mbar above atmospheric pressure and the other end of the tube is opened to atmospheric pressure, the air flow per unit area through the plug is in the range of 7.5 to 40 ml/min.mm2.

The invention relates to straws for the conservation of predetermined doses of liquid base substances, in particular biological substances, for instance pure or diluted animal semen or a conservation medium containing embryos.

It is known that such a straw is conventionally formed by a thin tube, having for instance an inner diameter of 1.6 or 2.5 mm and a length of 133 mm, and by a plug inserted in the thin tube.

In the filled condition, the plug is located in the vicinity of a first end of the tube and the dose of substance is located in the straw between the plug and the second end of the tube.

For filling the straw, the first end of the tube, close to the plug, is placed in communication with a vacuum source whereas the second end is placed in communication with a vessel containing the substance to introduce into the straw. The air initially contained between the plug and the second end is sucked through the plug whereas the substance makes way into the tube until it reaches the plug, into which it cannot goes through because the plug is liquid tight, by nature or on contact with a liquid.

The filled straw is in general cold store at a cryogenic temperature (conservation into liquid nitrogen), electrical cold (cold production by Peltier effect) or mechanical cold (cold production by compressor). In certain cases, where the conservation duration is short, the straw is simply kept at ambient temperature.

For emptying the straw, after thawing if necessary, the plug is slid towards the second end of the tube, as a piston, so that the dose of substance initially contained in the straw is expelled therefrom by the second end.

In general, straw plugs are of the tripartite type originally disclosed in French patent 995.878, to which British patent 669,265 is counterpart, that is formed by two pads of fibrous substance enclosing a powder capable of being transformed on contact with a liquid into an impermeable gel or paste adhering to the wall of the tube.

French patent application 2 787 011, to which U.S. Pat. No. 6,300,125 is counterpart, proposes to fit within the tube an insert which is a capillary tube, the diameter of the coaxial orifice in the insert being sufficiently small to stop the substance by capillary effect. The insert is used as a conventional plug.

Solutions have been already proposed for limiting or even eliminating the loss of substance owing to the absorption by the plug.

French patent applications 2 824 255 and 2 824 256, to which American patent applications US 2002/0183653 and US 2002/0188222 are counterparts, propose that the plug, in addition to the powder and the fibres, includes non-absorbent members, namely a core in thermoplastic material, coated with a sheath in braided yarns, and/or non-absorbent material in dispersed form in the powder.

European patent 0 873 726 proposes that the plug is a one-piece microporous hydrophobic cylinder. The hydrophobic nature of the one-piece plug makes it possible to stop the substance with no absorption.

Such a plug is very satisfactory in terms of the loss of substance but it is relatively difficult to manufacture.

The invention is directed to providing a straw with no or limited loss of substance into the plug while being simple, convenient and economic in manufacture and in use.

According to the invention, a straw is provided for the conservation of a predetermined dose of a liquid base substance, in particular a biological substance, comprising a tube and a plug consisting of a one-piece cylinder of microporous material, permeable to gases and liquid tight, said plug being inserted in said tube, said tube having a constant inner diameter and said plug being able to slide as a piston within said tube up to the end most remote from the original location of the plug; characterized in that said microporous material is a sintered self-sealing microporous material with at least a solid portion having a diameter matching the inner diameter of said tube, said solid portion being in direct contact with said tube, said plug having in the tube a permeability to gases when it is dry such that if one end of the tube is placed in communication with a pressure source 150 mbar above atmospheric pressure and the other end of the tube is opened to atmospheric pressure, the air flow per unit area through the plug is in the range of 7.5 to 40 ml/min.mm².

For instance, if the inner diameter of the tube is 1.6 mm (inner cross section of 2 mm²), the air flow rate through the plug would be in the range of 15 to 80 ml/min.

The determination of the airflow rate through the plug can be made with a conventional leakage detector machine or with a pneumatic circuit with a good precision pressure regulator (e.g. +/−2 mbar) and air flow rate meter (e.g. +/−1 ml/min).

Unlike the microporous plug of the straw disclosed in European patent 0 873 726, the microporous plug of the straw according to the invention is in a material which is sintered (and not fibrous) and self-sealing (and not hydrophobic).

In sintered self-sealing microporous materials, the self-sealing capacity is provided by the presence of a water absorptive component having a relatively high molecular weight and which can be dissolved in water very quickly. When a small amount of the water absorptive component dissolves in water, it forms a very high viscosity solution, like gel. The high viscosity solution or gel blocks the pores of the sintered self-sealing microporous material and prevents passage of liquid and gas.

While this may appear surprising, it has been found that the use of a plug in sintered self-sealing microporous material having the above mentioned permeability to air (air flow per surface unit) enables not only the filling of the straw in very good conditions, but also a very low absorption of the liquid by the plug.

This is probably because one of the main parameters influencing air flow through the plug is the pore size.

With an air flow per surface unit lower than 7.5 ml/min.mm² at 150 mbar, the filling would be so slow that with conventional straw filling machine, the straw would not be completely filled at the end of the conventional time for filling the straw (usually 0.2 to 3 s).

With an air flow per surface unit higher than 40 ml/min.mm² at 150 mbar, the pore size is liable to be so big that the water entry pressure is too low and the liquid enters the cylinder too easily and therefore the absorption rate would be very high.

With the above mentioned air flow per surface unit, the sintered self-sealing microporous material is capable of performing the transformation from a gas permeable to a liquid tight condition with a rate of liquid absorption which is much lower than in the plug with powder such as plugs of the tripartite type.

For instance, for a 0.25 ml straw (thin tube having an inner diameter of 1.6 mm and a length of 133 mm), the rate of liquid absorption can be 1 or 1.5%. The determination of the rate of liquid absorption is made by filling the straw with water from a vessel previously weighted and then expelling from the straw the water back in the vessel and then weighting the vessel. The difference in weight gives the weight of the water absorbed by the plug and consequently the volume of water absorbed by the plug.

The manufacture of sintered self-sealing microporous material, while being delicate in some aspects, is today very well known as explained for instance in column 10 to 12 of U.S. Pat. No. 6,808,908, and blocks of sintered self-sealing materials are now used in a variety of devices such as in pipette tips for preventing overflow (see for instance U.S. Pat. No. 5,156,811 and PCT application WO 02/36708).

Similar manufacturing processes and facilities can be used for manufacturing the plug of the straw according to the invention, of course with the details of the raw material blend (defined by particles size and shape) as well as the details of the method to process the raw material which are adapted (the air flow per surface unit is not optimized in the known blocks of sintered self-sealing microporous material).

It should be noted that it has already been proposed in the field of arterial blood sample collection for blood gas analysis to use a piston plug including a block of sintered self-sealing microporous material, for instance in U.S. Pat. No. 4,703,763.

In the device disclosed by this document, the block is surrounded by an exterior member made of Kraton interposed between the block and the tube and resiliently engaged with the tube to form a seal between the tube and the block.

The device disclosed in this document is not designed for the conservation of the blood sample. Instead, the blood sample is expelled from the tube directly after collection for analysis purposes. The Kraton in the plug is not able to withstand cryogenic freezing (a conventional straw is able to withstand temperatures in the range of −196° C. to 80° C.).

The plug in the straw according to the invention offers with respect to the plug disclosed in U.S. Pat. No. 4,703,763 not only the advantage of being far more temperature resistant but also the advantage of being more simple and convenient because the liquid tightness between the sintered self-sealing microporous material and the tube is provided by the mere direct contact between the solid portion of the cylinder and the tube.

The plug of the straw according to the invention also offers with respect to the plug in French patent application 2 787 011 or U.S. Pat. No. 6,300,125, the advantage of providing the required air flow with at least the portion of the cylinder in contact with the tube being solid and therefore reliable and less absorbing than the centrally hollow non-porous insert disclosed in this document.

The plug of the straw according to the invention also offers with respect to conventional tripartite plugs, because of its one-piece or inherently cohesive mechanical nature, the advantage of being more resistant to shocks or vibrations.

According to preferred features of the straw according to the invention, the plug has a pore size in the range of 10 to 50 μm and a pore volume in the range of 10 to 50%.

The above values of the pore size and the pore volume are determined with a mercury porosimeter device such as the one available from CE Instruments limited in the UK (www.ceinstruments.co.uk) under the name Pascal 140. Mercury porosimetry analysis is based on the intrusion of mercury into the solid material porous structure under controlled pressurization. The pore size is determined from the pressure necessary for penetration and the pore volume from the volume of penetrated mercury. In the present document, the range given for the pore size is the range of the peak value for the pore size distribution.

It has been found that the above ranges for the pore size and the pore volume enable excellent performance to be obtained.

Most preferably, the plug has a pore size in the range of 15 to 35 μm and a pore volume in the range of 25 to 50%.

With a pore size lower than 15 μm, the absorption of the liquid by the plug would be lower but the filling of the straw may be irregular and the liquid may even be prevented from entering into the plug. If the liquid does not enter the plug, there is a risk that the plug will not be liquid tight enough to prevent liquid nitrogen passing around the plug and reaching the substance to be conserved. This is a risk which is to avoided absolutely, because of possible cross-contamination through liquid nitrogen and because the liquid nitrogen trapped behind the plug is liable to expand strongly during thawing and cause the straw to explode.

With a pore size greater than 35 μm, there is a risk of leakage through the plug and the absorption rate would be too high.

With a pore volume lower than 25%, the airflow density would be very low and the plug may even not be porous and permeable any longer.

With a pore volume greater than 50%, the absorption rate would be too high.

According to preferred features of the straw according to the invention:
the plug is made of a thermoplastic and a self-sealing additive;
said thermoplastic is polyethylene;
said additive includes carboxyl methyl cellulose (CMC); and/or
the self-sealing additive is in the range of 5 to 20% by weight.

The choice of a thermoplastic, such as polyethylene, and of a self-sealing additive, such as CMC, is useful for providing the plug with the required elasticity to fit tightly within the tube without difficulty of insertion in the tube and with a good stability important for the storage of the assembled straws and for the ability to expand again during the thawing process so as to prevent leakage during or after thawing.

If the proportion of the self-sealing additive is greater than 20% by weight, the liquid would not penetrate the plug enough or even prevent the liquid from entering the plug, with the above-mentioned risk of having the liquid nitrogen passing around the plug.

Most preferably, the self-sealing additive is in the range of 8 to 12% by weight.

The choice of a self-sealing additive such as CMC in the range of 8 to 12% by weight has been found to provide an optimal penetration of the liquid into the plug.

If the proportion of self-sealing additive is lower than 8% by weight, the liquid would penetrate deeply into the plug and the absorption rate would be too high.

If the proportion of the self-sealing additive is greater than 12% by weight, there is a risk that on contact with a liquid, a gel effect would be visible on the side of the plug wetted by the liquid.

Additionally, with the proportion of self-sealing additive in the range of 8 to 12% by weight, the lubricant effect provided by the self-sealing additive when the plug is wetted because the surface of the plug is then covered with a very thin gel layer, provides an optimum lubricant effect useful for decreasing the force necessary to move the plug with respect to the tube.

According to further preferred features of the straw according to the invention the ratio between the inner diameter of the tube and the diameter of the solid portion of the plug when it is outside the tube is in the range of 80 to 95%.

This range provides a compression of the plug because of the insertion of the plug in the tube which is sufficiently high to prevent leakage during the filling, freezing, thawing and emptying steps and sufficiently low to prevent too big a change of the pore size and too big a force needed to empty the straw.

According to further preferred features of the straw according to the invention:
said plug includes exclusively said solid portion; and possibly
said plug has when inserted in the tube a length in the range of 2 to 5 mm.

Such a length for the plug is sufficiently high to prevent difficulties of introduction of the plug in the straw and to provide a safety margin with respect to the expected depth of penetration of the liquid into the plug, which is for instance 1 mm.

The plug is also sufficiently short for the airflow to be sufficient and for the friction with the tube to be compatible with the conventional forces for moving the plug so as to expel the substance from the tube (3 to 5 N and no peak value higher than 15 to 20 N).

In an another embodiment of the straw according to the invention, the plug includes said solid portion and two lateral portions.

With such an arrangement, the plug is able to slide within the tube more easily than if the plug was in contact with the tube over its entire length; and such an arrangement remains easy to manufacture according to the conventional methods for obtaining one-piece blocks of sintered self-sealing material.

The disclosure of the invention will now be continued with the description of example embodiments of straws according to the invention given hereafter in a non-limiting manner with reference to the appended drawings, in which:

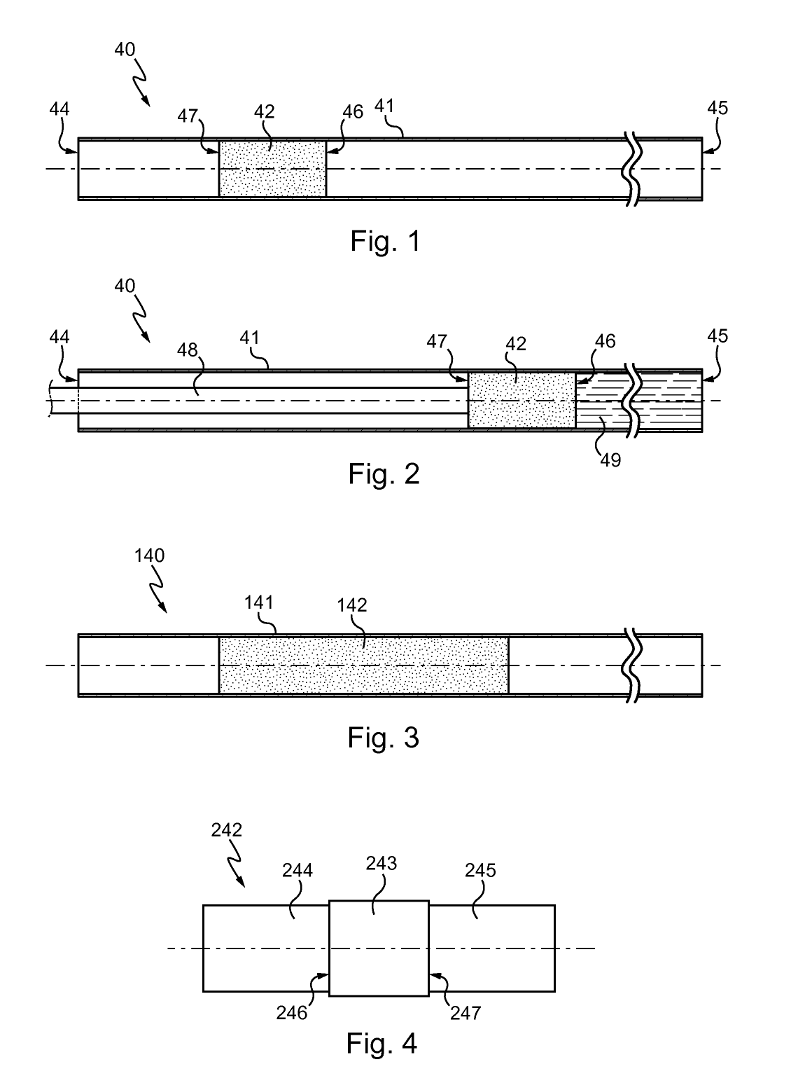

FIG. 1 is a schematic view in longitudinal cross-section of a straw according to the invention;

FIG. 2 is a view similar to FIG. 1 showing how the straw according to the invention is emptied;

FIG. 3 is a view similar to FIG. 1 for a variant embodiment where the plug is longer; and

FIG. 4 is a schematic view, enlarged with respect to FIGS. 1 to 3, of a variant embodiment of the plug.

The device 40 illustrated on FIG. 1 is a straw for the conservation of a predetermined dose of a liquid base substance, in particular a biological substance, for instance pure or diluted animal semen or a conservation medium containing embryos.

Straw 40 is formed by a tube 41 and a plug 42 which are cylindrical, that is having a constant diameter over their length.

Tube 41 is an extruded plastic material, with an inner diameter which is for example of 1.6 or 2.5 mm and a length of about 133 mm.

Plug 42 is a one-piece solid cylinder of a sintered self-sealing microporous material.

The diameter of plug 42, before introduction in tube 41, is slightly greater than the inner diameter of this tube, so that plug 42 fits tightly in tube 11. This provides a good seal between plug 42 and the inner wall of tube 41, thanks to the light compression resulting therefrom. Here, the diameter of plug 42 before introduction is 1.8 mm whereas the inner diameter of tube 41 is 1.60 mm.

The length of plug 42 is between 2 and 5 mm, here 3 mm.

Straw 40 is conventionally used.

In the initial condition, illustrated on FIG. 1, plug 42 is placed in the vicinity of end 44 of tube 41.

In the filled condition, the dose of liquid substance which must be preserved in straw 40 is placed between plug 42 and the end 45 of tube 41 most remote from plug 42.

For filling straw 40, end 44 is placed in communication with a vacuum source whereas end 45 is placed in communication with a vessel containing the substance to introduce into the straw.

The air initially contained between plug 42 and end 45 is sucked through plug 42 whereas the substance makes way in the tube until it reaches plug 42, by the end 46 thereof turned towards end 45 of tube 41, that is the end of plug 42 seen on the right on FIG. 1.

Plug 42 then stops the progression of the substance because on contact therewith at least the portion of plug 42 in the vicinity of end 46 assumes a wet condition.

The tube may be welded in the vicinity of one or both extremities 44 and 45 and is cold stored.

For emptying the straw, possibly after cutting off the welded end portions and thawing, a small rod 48 (FIG. 2) is introduced in tube 41 and comes to bear against end 47 of plug 42 (end turned towards end 44).

As shown on FIG. 2, this makes it possible to slide plug 42 in the manner of a piston towards end 45 or the corresponding end after cutting off the welded portion, and this causes expulsion of the dose of substance 49 which was introduced into the straw.

The straw 140 illustrated on FIG. 3 will now be disclosed. The same reference numerals as for straw 40 have been used with the addition of 100.

Straw 140 includes a tube 141 identical to tube 41 and a plug 142 identical to plug 42 except that it is longer, here 7 mm long instead of 3 mm.

Such a greater length is useful for certain self-sealing materials so as to provide a better sealing between the plug and the tube.

The plug 242 illustrated in FIG. 4 is identical to plug 142 except that it is in three portions with a central portion 243 having a diameter slightly greater than lateral portions 244 and 245 so that there is a shoulder 246 between lateral portion 244 and central portion 243 and a shoulder 247 between lateral portion 245 and central portion 243.

The diameter of central portion 243 is the same as the diameter of plug 42 or plug 142 so that plug 242 is in contact with a tube such as tube 41 or tube 141 by the outer surface of central portion 243.

Many other variant embodiments are feasible and it is to be recalled that the invention is not limited to the embodiments illustrated and disclosed.

1. Straw for the conservation of a predetermined dose of a liquid base substance, in particular a biological substance, comprising a tube (41; 141) and a plug (42; 142; 242) consisting of a one-piece cylinder of microporous material, permeable to gases and liquid tight, said plug being inserted in said tube, said tube (41; 141) having a constant inner diameter and said plug (42; 142; 242) being able to slide as a piston within said tube up to the end (45) most remote from the original location of the plug; wherein said microporous material is a sintered self-sealing microporous material with at least a solid portion (42; 142; 243) having a diameter matching the inner diameter of said tube (41; 141), said solid portion being in direct contact with said tube (41; 141), said plug having in the tube a permeability to gases when it is dry such that if one end of the tube is placed in communication with a pressure source 150 mbar above atmospheric pressure and the other end of the tube is opened to atmospheric pressure, the air flow per unit area through the plug is in the range of 7.5 to 40 ml/min.mm².

2. Straw according to claim 1, wherein the plug (42; 142; 242) has a pore size in the range of 10 to 50 μm and a pore volume in the range of 10 to 50%.

3. Straw according to claim 2, wherein the plug (42; 142; 242) has a pore size in the range of 15 to 35 μm and a pore volume in the range of 25 to 50%.

4. Straw according to claim 1, wherein the plug (42; 142; 242) is made of a thermoplastic and a self-sealing additive.

5. Straw according to claim 4, wherein said thermoplastic is polyethylene.

6. Straw according to claim 4, wherein said additive includes carboxyl methyl cellulose (CMC).

7. Straw according to claim 4, wherein the self-sealing additive is in the range of 5 to 20% by weight.

8. Straw according to claim 7, wherein the self-sealing additive is in the range of 8 to 12% by weight.

9. Straw according to claim 1, wherein the ratio between the inner diameter of the tube (41; 141) and the diameter of the solid portion of the plug (42; 142; 243) when it is outside the tube is in the range of 80 to 95%.

10. Straw according to claim 1, wherein said plug includes exclusively said solid portion (42; 142).

11. Straw according to claim 10, wherein said plug has when inserted in the tube a length in the range of 2 to 5 mm.

12. Straw according to claim 1, wherein said plug includes said solid portion (243) and two lateral portions (244, 245).
